SEQUENCE LISTING (10 sequences)

SEQ ID NO: 1 - DNA, 1296 bp, synthetic construct
  misc_feature 1..1296: note = FOXP3 polynucleotide sequence
  source 1..1296: mol_type = other DNA
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 2 - DNA, 1352 bp, synthetic construct
  misc_feature 1..1352: note = FOXP3 polynucleotide sequence
  source 1..1352: mol_type = other DNA
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 3 - protein, 431 aa, synthetic construct
  REGION 1..431: note = FOXP3 polypeptide sequence - UniProtKB accession Q9BZS1
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 4 - protein, 441 aa, synthetic construct
  REGION 1..441: note = FOXP3 polypeptide sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 5 - protein, 452 aa, synthetic construct
  REGION 1..452: note = FOXP3-2A polypeptide sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPGATNFSLLKQAGDVEENPGPS

SEQ ID NO: 6 - protein, 462 aa, synthetic construct
  REGION 1..462: note = FOXP3-2A polypeptide sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEENGATNFSLLKQAGDVEENPGPS

SEQ ID NO: 7 - protein, 19 aa, synthetic construct
  REGION 1..19: note = MBP111-129
LSRFSWGAEGQRPGFGYGG

SEQ ID NO: 8 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 9 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 10 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

(19) United States
(12) Patent Application Publication
(10) Pub. No.: US 2025/0283039 A1
(43) Pub. Date: Sep. 11, 2025

(54) METHOD FOR MAINTAINING SUPPRESSIVE ACTIVITY OF REGULATORY T CELLS

(71) Applicant: QUELL THERAPEUTICS LIMITED, London (GB)

(72) Inventor: Jenny MCGOVERN, London (GB)

(21) Appl. No.: 18/859,911

(22) PCT Filed: May 5, 2023
(86) PCT No.: PCT/GB2023/051200

§ 371 Date: Oct. 24, 2024

(30) Foreign Application Priority Data
May 5, 2022 (GB) 2206570.0

(51) Int. Cl. C12N 5/0783; A61K 40/11; A61K 40/31; A61K 40/41; C07K 14/47

(52) U.S. Cl. CPC C12N 5/0637; A61K 40/11; A61K 40/31; A61K 40/416; C07K 14/4705; C12N 2501/2301; C12N 2501/2306; C12N 2501/25; C12N 2510/00

(57) ABSTRACT

The present invention provides a method for maintaining the ability of a regulatory T cell (Treg) to suppress immune responses under proinflammatory conditions comprising introducing a polynucleotide encoding a FOXP3 polypeptide into the Treg.

### FIELD OF THE INVENTION

The present invention relates to a method for maintaining the ability of regulatory T cells (Tregs) to suppress immune responses when under proinflammatory conditions, particularly CAR Tregs. In particular, the present invention relates to a method of increasing FOXP3 expression in Tregs. The present invention further relates to an engineered Treg provided by the method of the invention and to methods and uses of such an engineered Treg.

### BACKGROUND TO THE INVENTION

In autoimmune and inflammatory central nervous system (CNS) diseases, the immune system attacks self-antigens. For example, in Multiple Sclerosis (MS), the most common neurological disorder among young adults, the immune system attacks the myelin sheath of neurons of the central nervous system.

Current treatments for autoimmune and inflammatory CNS diseases generally suppress the immune system. For example, one treatment includes transplantation of bone marrow along with administration of cytostatics and immunosupressive drugs. Autologous haematopoietic stem cell transplantation can have lasting beneficial effects for some subjects, but the procedure requires aggressive myelo-ablative conditioning which is associated with substantial toxicity and risk.

Although several disease-modifying treatments (DMTs) have been approved to reduce the frequency of clinical relapses, most patients continue to clinically deteriorate under current therapy schedules. Neither DMTs nor stem cell transplantation can mediate CNS-specific suppression of the immunopathology of autoimmune and inflammatory CNS diseases.

Currently, effective treatments for autoimmune and inflammatory CNS diseases do not exist. Treatment is focused on merely reducing its symptoms, usually by general suppression of the immune system. There is a need for a therapy which specifically targets local immune responses associated with onset and progression of CNS disease.

Regulatory T cells (Tregs) are a type of T cell that modulates the activity of the immune system. Generally, Tregs are immunosuppressive, down-regulating immune responses to stimuli. In particular, Tregs suppress induction and proliferation of conventional T cells, some types of which are directly involved in immune responses (e.g. cytotoxic T cells). The suppressive effect of Tregs can be directed towards specific antigens by expression of recombinant T cell receptor (TCR) constructs that recognise peptides matching epitopes found within the antigen in question. Similarly, the suppressive effect of Tregs can be directed towards specific targets by expression of chimeric antigen receptors (CARs) that recognise antigens expressed on the surface of target cells. Conventional T cells can be differentiated towards a regulatory phenotype ex-vivo by expressing FOXP3 in said cells.

Regulatory T cells (Tregs) can modulate immune responses through multiple mechanisms; therefore, their therapeutic use has been proposed to induce immune tolerance in transplantation, autoimmune diseases, and chronic inflammatory conditions. FOXP3 is considered the master transcription factor of Tregs, driving expression of CD25, CTLA-4 and repression of pro-inflammatory cytokines such as IL-2. It has been shown that some cells that bear all the hallmarks of Tregs can, under certain conditions, lose expression of FOXP3 and acquire pathogenic features, such as expression of effector cytokines. This outcome would be particularly problematic in the context of antigen-specific Treg therapies—posing a significant safety issue.

### SUMMARY OF THE INVENTION

The present inventors have surprisingly found that exogenous FOXP3 expression in regulatory T cell (Tregs) (which already express endogenous FOXP3) allows maintenance of their regulatory function when exposed to proinflammatory conditions.

Accordingly, the invention provides a method for maintaining the ability of regulatory T cells (Tregs) to suppress immune responses when exposed to proinflammatory conditions.

The Tregs of the present invention are natural Tregs or induced Tregs which developed from conventional T cells. For example, the Tregs of the present invention are natural Tregs or induced Tregs which developed from conventional T cells in vivo. Suitable Treg cells include thymus-derived, natural Treg (nTreg) cells and peripherally generated, induced Treg (iTreg) cells. In other words, the Tregs of the present invention express endogenous FOXP3. Surprisingly, the present inventors have determined that increasing FOXP3 expression in Tregs which already express endogenous FOXP3 (e.g. by introducing exogenous FOXP3) maintains the regulatory function of the Tregs when exposed to proinflammatory conditions at similar levels to corresponding Tregs which are not exposed to proinflammatory conditions (e.g. which are cultured under non-inflammatory conditions). In contrast to this, Tregs with endogenous levels of FOXP3 (e.g., Tregs without exogenous FOXP3) have been shown to lose their ability to suppress under proinflammatory conditions. The presence of exogenous FOXP3 in Tregs which already express endogenous FOXP3 therefore appears to provide the Tregs with functional stability as compared to cells with only endogenous FOXP3 expression (particularly natural or wildtype endogenous FOXP3 expression) which cells appear to be susceptible to function reduction under proinflammatory conditions. Thus, alternatively viewed, the present invention further provides a method of maintaining functional stability of a Treg under proinflammatory conditions comprising the step of increasing expression of FOXP3 (e.g. expressing exogenous FOXP3) in the Treg or a method of maintaining suppressive function in a Treg under proinflammatory conditions comprising the step of increasing expression of FOXP3 (e.g. expressing exogenous FOXP3) in the Treg.

In a preferred embodiment, the Tregs of the present invention are natural Tregs.

The invention provides a method for maintaining the ability of a Treg to suppress immune responses under proinflammatory conditions comprising increasing FOXP3 expression in the Treg.

In some embodiments of the invention, FOXP3 expression is increased by introducing into the Tregs a polynucleotide encoding a FOXP3 protein.

In some embodiments of the invention, the method for maintaining the ability of regulatory T cells (Tregs) to suppress immune responses under proinflammatory conditions comprises:
(a) isolating a Treg from a cell population; and
(b) increasing FOXP3 expression in said Treg.

Suitably, the Treg may refer to a population of Tregs (i.e. a plurality of Tregs).

The methods of the invention may include an additional step of exposing the Treg cells to proinflammatory conditions.

The invention also provides an engineered Treg obtainable or obtained by the method of the invention.

The invention also provides a pharmaceutical composition comprising an engineered Treg of the invention.

The invention also provides an engineered Treg of the invention or a pharmaceutical composition of the invention for use in prevention and/or treatment of a disease.

The invention also provides the use of an engineered Treg of the invention in the manufacture of a medicament.

The invention also provides a method for prevention and/or treatment of a disease comprising administering to a subject an engineered Treg or a composition of the invention.

The invention also provides the use of a polynucleotide encoding a FOXP3 polypeptide to maintain the ability of a regulatory T cell (Treg) to suppress immune responses under proinflammatory conditions.

The invention further provides use of a nucleic acid molecule comprising a nucleotide sequence encoding FOXP3 for maintaining the suppressive function or functional stability of a Treg when exposed to proinflammatory conditions, wherein said Treg comprises said nucleic acid molecule.

### DESCRIPTION OF THE FIGURES

FIG. 1 shows the outline method of an instability assay, used to determine functional stability of a Treg. Cells are incubated in either control conditions or in the presence of three proinflammatory cytokines.

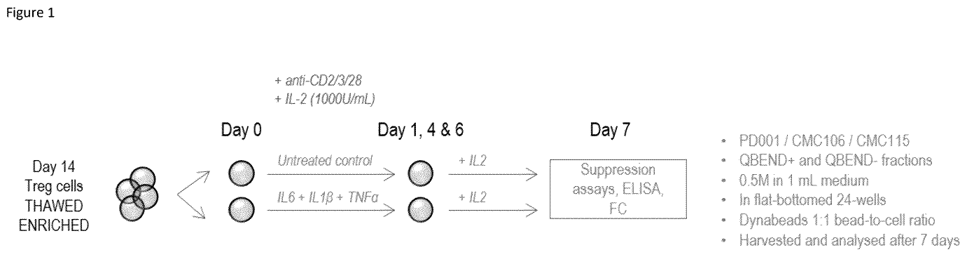

FIG. 2 shows the construct design used in the instability assay. Two constructs were used, namely construct VIII which encodes only a CAR (an HLA-A2 CAR) and construct I which encodes FOXP3 and a CAR (encoding polynucleotide sequences separated by a 2A self-cleaving peptide). FIG. 2 further shows the instability assay.

FIG. 3 shows the results of an instability assay performed using a Treg population transduced with a polynucleotide encoding construct I which was exposed to proinflammatory conditions. FOXP3 expression was determined in transduced and non-transduced cells and the cells were used in a suppression assay. Transduced cells had a higher % of FOXP3 as compared to non-transduced cells and were able to suppress even after exposure to proinflammatory conditions as compared to the non-transduced cells which were not capable of suppression after exposure to proinflammatory conditions.

FIG. 4 shows the ability of Tregs transduced with either construct I or construct VIII to suppress when exposed to proinflammatory conditions. Tregs expressing construct I (and thus expressing exogenous FOXP3 and a CAR) maintain their suppressive effect after exposure to proinflammatory cytokines. The clear circles show the construct I comprising cells when exposed to control conditions and the grey circles show construct I comprising cells after exposure to proinflammatory conditions, where the maintenance of suppressive function can be seen. In contrast to this, Tregs expressing construct VIII (no exogenous FOXP3, CAR only), cannot suppress after exposure to proinflammatory conditions and have similar low levels of suppression comparable to non-transduced cells.

### DETAILED DESCRIPTION

The invention provides a method for maintaining the ability of a regulatory T cell (Tregs) to suppress immune responses when exposed to proinflammatory conditions comprising increasing FOXP3 expression the Treg (e.g. by expressing exogenous FOXP3).

### Regulatory T Cells

The term “regulatory T cell” (Treg) means a T cell which expresses the markers CD4, CD25 and FOXP3 (CD4⁺CD25⁺FOXP3⁺). Tregs may be identified using the cell surface markers CD4 and CD25 in the absence of or in combination with low-level expression of the surface protein CD127 (CD4⁺CD25⁺CD127⁻ or CD4⁺CD25⁺CD127^{low}). Tregs may also express on the cell surface high levels of CTLA-4 (cytotoxic T-lymphocyte associated molecule-4) or GITR (glucocorticoid-induced TNF receptor). Unlike conventional T cells, Tregs do not produce IL-2 and are therefore anergic at baseline.

The term “natural Treg” means a thymus-derived Treg. Natural Tregs are CD4⁺CD25⁺FOXP3⁺Helios⁺Neuropilin1⁺. The term “natural Treg” distinguishes thymus-derived Tregs from “induced Tregs”, which develop from conventional T cells outside the thymus. Compared with induced Tregs, natural Tregs have higher expression of PD-1 (programmed cell death-1, pdcd1), neuropilin 1 (Nrp1), Helios (Ikzf2), and CD73. Natural Tregs may be distinguished from induced Tregs on the basis of the expression of Helios protein or Neuropilin 1 (Nrp1) individually.

As used herein, the term “induced regulatory T cell” (iTreg) means a CD4⁺CD25⁺FOXP3⁺Helios⁻Neuropilin 1⁻ T cell which develops from mature CD4+ conventional T cells outside of the thymus. For example, iTregs can be induced in vitro from CD4+CD25−FOXP3− cells in the presence of IL-2 and TGF-β.

Suitably, the Treg expresses FOXP3 from the endogenous FoxP3 gene of the cell.

Suitably, the Treg may be a CD4⁺CD25⁺FOXP3⁺ Treg.

Suitably, the Treg may be a CD4⁺CD25⁺CD127⁻ Treg.

Suitably, the Treg may be a CD4⁺CD25⁺CD127^{low} Treg.

Suitably, the Treg may be a CD4⁺CD25⁺CD127⁻CD45RA⁺ Treg.

Suitably, the Treg may be a CD4⁺CD25⁺CD127^{low}CD45RA⁺ Treg

Suitably, the Treg may be a CD4⁺CD25⁺FOXP3⁺CD127⁻ Treg.

Suitably, the Treg may be a CD4⁺CD25⁺FOXP3⁺CD127^{low} Treg.

Suitably, the Treg is a CD4⁺CD25⁺FOXP3⁺Helios⁺ Treg.

Suitably, the Treg is a CD4⁺CD25⁺FOXP3⁺Neuropilin 1⁺ Treg.

Suitably, the Treg is a CD4⁺CD25⁺FOXP3⁺Helios⁺Neuropilin 1⁺ Treg.

Suitably, the Treg is a human Treg. Suitably the Treg is a human Treg and the FOXP3 is human FOXP3.

Typically, “a corresponding Treg” as used herein has the same or similar phenotype to a Treg of interest or of comparison, but may differ in either the genetic modification made (typically herein the increased level of FOXP3) or in the conditions to which the Treg has been exposed (typically herein proinflammatory vs non-inflammatory conditions). Same or similar phenotype means that the Treg may express the same or similar markers (e.g. may have at least 80, 90 or 95% of their marker molecules in common at the same or similar amounts, e.g. at least 70, 80 or 90% of the levels).

In one embodiment, the methods of the invention may particularly be used to stabilise the function, maintain the suppressive effect or prevent loss of the suppressive function under proinflammatory conditions as described herein of a Treg or Treg population which has an unstable phenotype. Particularly, the expression of an exogenous FOXP3 within such unstable Tregs may have a disproportionately positive effect on the maintenance of function (and thus the prevention of loss of function) as described herein. Thus, particularly, the Tregs used within the present invention may express reduced levels of Helios and/or may have a TSDR which is more than 50% methylated, e.g., is more than 60, 70, or 80% methylated. Demethylated TSDRs are associated with endogenous FOXP3 expression and thus methylation of the TSDR is associated with a reduction in endogenous FOXP3 expression and poorer Treg stability. Alternatively viewed, an unstable or less stable Treg population as may be used herein may be a population where more than 30, 40 or 50% of Treg cells within the population may have a methylated TSDR. Methylation status of the TSDR can be determined by any well known method of the art including bisulphite sequencing.

Alternatively, a Treg or population of Tregs which has a reduced stability or unstable phenotype may be characterised by expression of IL2. Tregs with a stable phenotype typically do not express IL2, and thus expression of any IL2 by a Treg may be indicative of an unstable phenotype. Thus, a Treg or a population of Tregs as used herein may express IL2, e.g. may express 10, 20, 30, 40 or 50% more IL2 than a Treg cell with a stable phenotype. It will be appreciated by a skilled person that not all Treg cells within a Treg population may express IL2 for the population to be considered as having an unstable phenotype or as being unstable (e.g. having reduced stability), although typically, at least 10, 20, 30, 40 or 50% of the Tregs within a Treg population may express IL2. As discussed above, expression of exogenous FOXP3 within such cells can result in a stabilisation of the phenotype (e.g. reduction in expression of IL2 or reversion to a cell which does not express IL2) and a cell or a population of cells which is/are capable of maintaining suppressive function under proinflammatory conditions.

The Treg or population of Tregs used herein may be obtained from a patient, typically a human patient. Such a patient Treg or population of Tregs may have a reduced or unstable phenotype as compared to Treg cells which may be isolated from a healthy donor or subject. Particularly, the Treg or population of Tregs may be obtained from a patient who has received a transplant, e.g. a liver, islet or kidney transplant, or from a patient having a condition associated with undesirable inflammation e.g. an autoimmune condition such as Type I Diabetes, or IBD.

The term “Tconv cells”, meaning conventional T cells, refers to T cells that are not Tregs.

In one aspect, the Treg of the present invention may be derived from a stem cell. In particular, the Treg of the present invention may be derived from a stem cell in vitro.

In another aspect, the cell is a progenitor cell.

As used herein, the term “stem cell” means an undifferentiated cell which is capable of indefinitely giving rise to more stem cells of the same type, and from which other, specialised cells may arise by differentiation. Stem cells are multipotent. Stem cells may be for example, embryonic stem cells or adult stem cells.

As used herein, the term “progenitor cell” means a cell which is able to differentiate to form one or more types of cells but has limited self-renewal in vitro.

Suitably, the cell is capable of being differentiated into a T cell, such as a Treg.

Suitably, the cell has the ability to differentiate into a T cell, which expresses FOXP3 such as a Treg.

Suitably, the cell may be an embryonic stem cell (ESC). Suitably, the cell is a haematopoietic stem cell or haematopoietic progenitor cell. Suitably, the cell is an induced pluripotent stem cell (iPSC). Suitably, the cell may be obtained from umbilical cord blood. Suitably, the cell may be obtained from adult peripheral blood.

In some aspects, hematopoietic stem and progenitor cell (HSPCs) may be obtained from umbilical cord blood. Cord blood can be harvested according to techniques known in the art (e.g., U.S. Pat. Nos. 7,147,626 and 7,131,958 which are incorporated herein by reference).

In one aspect, HSPCs may be obtained from pluripotent stem cell sources, e.g., induced pluripotent stem cells (iPSCs) and embryonic stem cells (ESCs).

As used herein, the term “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(−) cells includes haematopoietic stem cells and hematopoietic progenitor cells.

HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton's jelly, placenta, fetal blood, fetal liver, or fetal spleen. In particular embodiments, harvesting a sufficient quantity of HSPCs for use in therapeutic applications may require mobilizing the stem and progenitor cells in the subject.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state.

As used herein, the term “programming” refers to a method of decreasing the potency of a cell or differentiating the cell to a more differentiated state.

### Immune Responses

The expression “maintaining the ability to suppress immune responses” means to maintain the suppressive effect of a Treg (or population of such Tregs) on an immune response under proinflammatory conditions (e.g. after or during exposure to proinflammatory conditions), at a similar level as a corresponding Treg (particularly, a Treg which has been modified in the same manner or comprises the same modifications (or population of such Tregs) e.g., to express an increased level of FOXP3 (particularly from an exogenous nucleic acid molecule or polynucleotide)), but which has not been exposed to proinflammatory conditions (e.g. which has been cultured under non-inflammatory conditions, e.g. in the absence of IFNγ, IL1β and/or IL6). A “similar level” as used herein refers to the suppressive function being no more than 10, 20, 30, 40 or 50% different (decreased or increased) to that of the corresponding Treg when not exposed to proinflammatory conditions. Particularly, a similar level, may be the same level. The expression “maintaining the ability to suppress immune responses” is used interchangeably herein with the expressions “maintaining functional stability” and “maintaining suppressive function”. The maintenance of suppressive function/functional stability/the ability to suppress immune responses is typically when the Tregs are exposed/have been exposed to proinflammatory conditions or are under proinflammatory conditions.

Alternatively viewed, a Treg comprising an increased level of FOXP3 (e.g., comprising an exogenous polynucleotide encoding FOXP3) may have an increased ability to suppress immune responses when compared to a Treg which does not have an increased level of FOXP3 and particularly a Treg which does not comprise an exogenous polynucleotide encoding FOXP3, when under or exposed to proinflammatory conditions, for example a non-transduced Treg or a Treg transduced with a construct which does not encode FOXP3. As discussed further below, the inventors have shown that cells which do not comprise exogenous FOXP3 lose their ability to suppress when exposed to proinflammatory conditions, whereas Tregs comprising exogenous FOXP3 are capable of suppressive function at a similar level under proinflammatory conditions to Tregs which have been cultured under non-inflammatory conditions (e.g. in media with IL2). The increased ability of a Treg as described herein to suppress immune responses as compared to a Treg which does not have an increased level of FOXP3 may be an increase of at least 20, 30, 40, 50, 60, 70, 80 or 90%.

In accordance with this alternatively viewed embodiment, the invention also provides a method for increasing the ability of a regulatory T cell (Treg) to suppress immune responses under proinflammatory conditions comprising introducing a polynucleotide encoding a FOXP3 polypeptide into the Treg, as compared to a Treg which does not have an increased level of FOXP3 (e.g. as compared to a Treg with endogenous levels of FOXP3, or as compared to a Treg which has not been modified to have increased levels of FOXP3).

Alternatively viewed, the present invention provides a method of preventing loss of suppressive function in a Treg cell when exposed to proinflammatory conditions, comprising expressing an increased level of FOXP3 in the Treg cell (particularly from an exogenous nucleic acid molecule comprising a polynucleotide sequence encoding FOXP3). The method (and methods of the invention as described herein) may further comprise a step of incubating the Treg cell under proinflammatory conditions, e.g. incubating with any one of IL-6, IFNγ and/or IL1β. Particularly, as described herein, the Treg may be engineered to comprise a nucleic acid molecule that expresses FOXP3 and a CAR, particularly where the nucleotide sequence encoding FOXP3 is 5′ to the nucleotide sequence encoding the CAR. Further, the Treg cell may be CD45RA+, particularly CD4+CD25+CD127loCD45RA+.

Loss of suppressive function as used herein (also referred to as loss of functional stability), refers to a loss of suppressive function of at least 10, 20, 30, 40, 50, 60, 70, 80 or 90%. Typically, such loss of suppressive function may occur, as discussed above, when Treg cells are not engineered to have increased expression of FOXP3 (particularly are not engineered to have an exogenous nucleic acid encoding FOXP3). Prevention of loss of suppressive function may result in an engineered Treg having a similar or the same suppressive function (e.g. a suppressive function at least 50, 60, 70, 80, 90, 95 or 99% similar) under proinflammatory conditions as the same engineered cell under non-inflammatory conditions.

“Exogenous” as defined herein means the expression of a protein within a cell of the invention from a polynucleotide sequence that has been introduced to the cell (i.e., not from a polynucleotide sequence originally present within the cell, i.e., an endogenous polynucleotide sequence).

The term “immune response” refers to a number of physiological and cellular effects facilitated by the immune system in response to a stimulus such as a pathogen or an autoantigen. Examples of such effects include increased proliferation of Tconv cells and secretion of cytokines. Any such effects may be used as indicators of the strength of an immune response. A relatively weaker immune response by Tconv in the presence of modified Tregs compared to non-modified Treg would indicate a relative enhancement of the modified Tregs to suppress immune responses. For example, a relative decrease in cytokine secretion would be indicative of a weaker immune response, and thus an enhancement of the ability of Tregs to suppress immune responses. Alternatively, a similar immune response by Tconv in the presence of modified Tregs exposed or under proinflammatory conditions as compared to modified Tregs which have not been exposed to proinflammatory conditions (or have only been exposed to non-inflammatory conditions) would indicate a maintenance of the modified Tregs to suppress immune responses. As discussed above, “similar” as used herein refers to the function being no more than 10, 20, 30, 40 or 50% different (decreased or increased) to that of the corresponding Treg when not exposed to proinflammatory conditions (or when only exposed to non-inflammatory conditions).

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs described herein having increased FOXP3 expression may proliferate 5, 10, 15, 20, 25, 30, 35 or 40% less than the same Tconv cells co-cultured with corresponding Tregs that do not have increased FOXP3 expression after exposure of the Tregs to proinflammatory conditions, or alternatively viewed, antigen specific Tconv cells co-cultured with Tregs as described herein having increased FOXP3 expression and exposed to proinflammatory conditions may proliferate at similar levels as compared to antigen-specific Tconv cells co-cultured with corresponding Tregs having increased FOXP3 expression which have not been exposed to proinflammatory conditions (or have only been exposed to non-inflammatory conditions).

Antigen-specific Tconv cells co-cultured with Tregs as described herein having increased FOXP3 expression may show a reduction of effector cytokine that is at least 10%, at least 20%, at least 30%, at least 40%, at least 50%, or at least 60% greater than corresponding Tconv cells co-cultured with corresponding Tregs that do not have increased FOXP3 expression after exposure of the Tregs to proinflammatory conditions or alternatively viewed, antigen-specific Tconv cells co-cultured with Tregs having increased FOXP3 expression and exposed to proinflammatory conditions may show effector cytokines at similar levels as compared to antigen-specific Tconv cells co-cultured with corresponding Tregs having increased FOXP3 expression which have not been exposed to proinflammatory conditions (or which have only been exposed to non-inflammatory conditions).

Antigen-specific Tconv cells co-cultured with Tregs as described herein having increased FOXP3 expression may produce 10%, 20%, 30%, 40%, 50%, 60% or less effector cytokine than corresponding Tconv cells co-cultured with corresponding Tregs that do not have increased FOXP3 expression after exposure of the Tregs to proinflammatory conditions or alternatively viewed, antigen-specific Tconv cells co-cultured with Tregs as described herein having increased FOXP3 expression and exposed to proinflammatory conditions may produce similar levels of effector cytokines as compared to antigen-specific Tconv cells co-cultured with corresponding Tregs having increased FOXP3 expression which have not been exposed to proinflammatory conditions (or which have only been exposed to non-inflammatory conditions).

The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Antigen-specific Tconv cells co-cultured with Tregs of the invention having increased FOXP3 expression may achieve suppression of IL-2 production at ½, ¼, ⅛, 1/10 or 1/20 the cell number of corresponding Tregs that do not have increased FOXP3 expression.

### Proinflammatory Conditions

“Proinflammatory conditions” as used herein, refers to conditions which would be capable of inducing, causing, promoting or prolonging inflammation in a subject. Proinflammatory conditions may be identified by the presence of one or more proinflammatory cytokines.

The exposure of a Treg cell as described herein to proinflammatory conditions, typically represented by the presence of one or more proinflammatory cytokines, may be for any period of time. Exposure of the Treg to proinflammatory conditions may occur in vitro (e.g. by the culture of Tregs in the presence of one of more proinflammatory cytokines, which may be exogenously added to culture or which may be produced by a cell during culture, particularly during co-culture) or in vivo (e.g. when the Tregs reach a proinflammatory environment). The one or more proinflammatory cytokines may be present in any amount but will typically be present in an amount that would be capable of exerting an effect on a non-transduced Treg (e.g. at least 1, 5, 10, 50 or 100 pg/ml). Thus, the methods of the invention may be in vitro, in vivo (e.g. after administration of a Treg to a patient, e.g. a human patient, having inflammation) or may comprise both in vitro and in vivo steps (e.g. engineering of the Tregs and/or any culture under or exposure to proinflammatory conditions may occur in vitro (or ex vivo) and/or in vivo).

As discussed above, exposure of a Treg cell to proinflammatory conditions may be for any period of time, but is typically for an extended period of time, particularly for at least 2, 6, 12, 24, 48, or 72 hours, and more particularly for at least 4, 5, 6 or 7 days. Typically, in vivo, proinflammatory conditions may increase over time and levels of proinflammatory cytokines may increase over an extended period of time. Thus, particularly in an in vivo setting, a Treg may be considered to be exposed to proinflammatory conditions if present at a site of where proinflammatory cytokines are present, for an extended period of time, e.g. for more than 1, 2, 3, 4, 5, 6 or 7 days. The Treg (or population of Tregs) as described herein may demonstrate a maintenance of suppressive effect after exposure to proinflammatory conditions (e.g. at least 1, 2, 6, 12 or 24 hours after), or during exposure to proinflammatory conditions (e.g. whilst the Treg is under or being exposed to proinflammatory conditions). “When exposed” to proinflammatory conditions includes prior exposure and current exposure.

Proinflammatory cytokines are molecules that maybe secreted from immune cells such as helper T cells and/or macrophages and that may promote inflammation in a subject or host. Proinflammatory cytokines include IL1β, IL6, IL12, IL18, IFNγ, GM-CSF and TNFα. Particularly, proinflammatory conditions, include the presence of one or more proinflammatory cytokines (e.g. at least 1, 2 or 3 proinflammatory cytokines), more particularly, anyone or more of IL1β, IL6 and TNFα, e.g. having a sequence of SEQ ID NO. 8, SEQ ID NO. 9 or SEQ ID NO. 10, respectively, or a functional variant thereof, typically having at least 80, 90 or 95% sequence identity thereto.

```
IL1β
(SEQ ID NO. 8)
                10 20 38 40 50
MAEVPELASE MMAYYSGNED DLFFEADGPK QMKCSFQDLD LCPLDGGIQL
                60 70 80 90 100
RISDHHYSKG FRQAASVVVA MDKLRKMLVP CPQTFQENDL STEFPFIFEE
              110 120 130 140 150
EPIFFDTWDN EAYVHDAPVR SLNCTLRDSQ QKSLVMSGPY ELKALHLQGQ
              160 170 180 190 200
DMEQQVVFSM SFVQGEESND KIPVALGLKE KNLYLSCVLK DDKPTLQLES
              210 220 230 240 250
VDPKNYPKKK MEKRFVENKI EINNKLEFES AQFPNWYIST SQAENMPVFL
              260
GGTKGGQDIT DFTMQFVSS
IL6
(SEQ ID NO. 9)
                10 20 38 40 50
MNSFSTSAFG PVAFSLGLLL VLPAAFPAPV PPGEDSKDVA APHRQPLTSS
                60 70 80 90 100
ERIDKQTRYI LDGISALRKE TCNKSNMCES SKEALAENNL NLPKMAEKDG
              110 120 130 140 150
CFQSGFNEET CLVKIITGLL EFEVYLEYLQ NRFESSEEQA RAVQMSTKVL
              160 170 180 190 200
IQFLQKKAKN LDATTTPDPT TNASLLTKLQ AQNQWLQDMT THLILASFKE
              210
FLQSSLRALR QM
TNFalpha
(SEQ ID NO. 10)
                10 20 38 40 50
MSTESMIRDV ELAEEALPKK TGGPQGSRRC LFLSEFSFLI VAGATTLFCL
                60 70 80 90 100
LHFGVIGPQR EEFPRDLSLI SPLAQAVRSS SRTPSDKAVA HVVANPQAEG
              110 120 130 140 150
QLQWLNRRAN ALLANGVELR DNQLVVPSEG LYLIYSQVLF KGQGCPSTHV
              160 170 180 190 200
LLTHTISRIA VSYQTKVNLL SAIKSPCQRE TPEGAEAKPW YEPIYLGGVF
              210 220 230
QLEKGDRLSA EINRPDYLDF AESGQVYFGI IAL
```

Typically, a Treg exposed to proinflammatory conditions as defined herein is exposed to any one or more of IL1B, TNFα and IL6 for at least 3 days, and particularly is exposed to all of IL1B, TNFα and IL6 for at least 3 days.

A Treg which is not exposed to proinflammatory conditions, may not have been cultured in vitro or exposed in vivo to one or more proinflammatory cytokines for an extended period of time, for example for more than 2, 6, 12, 24, 48, or 72 hours. Such a Treg may be referred to as being only exposed to non-inflammatory conditions. Typically, a Treg which is not exposed to proinflammatory conditions or which has only been exposed to non-inflammatory conditions will not have been incubated or cultured with a proinflammatory cytokine, e.g. with any one or more of IL6, TNFα and/or IL1B and typically with any of IL6, TNFα and IL1B.

### FOXP3

“FOXP3” is the abbreviated name of the forkhead box P3 protein. FOXP3 is a member of the FOX protein family of transcription factors and functions as a master regulator of the regulatory pathway in the development and function of regulatory T cells.

“Increasing FOXP3 expression” means to increase the levels of FOXP3 mRNA and/or protein in a Treg (or population of such Tregs) in comparison to a corresponding Treg which has not been modified (or population of such Tregs). For example, the level of FOXP3 mRNA and/or protein in a Treg modified as described herein (or a population of such Tregs) may be increased to at least 1.5-fold, at least 2-fold, at least 5-fold, at least 10-fold, at least 50-fold, at least 100-fold, at least 150-fold greater than the level in a corresponding Treg which has not been modified (or population of such Tregs).

Suitably, the level of FOXP3 mRNA and/or protein in a Treg modified as described herein (or a population of such Tregs) may be increased to at least 1.5-fold greater than the level in a corresponding Treg which has not been modified as described herein (or population of such Tregs).

Suitably, the level of FOXP3 mRNA and/or protein in a Treg modified as described herein (or a population of such Tregs) may be increased to at least 2-fold greater than the level in a corresponding Treg which has not been modified as described herein (or population of such Tregs).

Suitably, the level of FOXP3 mRNA and/or protein in a Treg modified as described herein (or a population of such Tregs) may be increased to at least 5-fold greater than the level in a corresponding Treg which has not been modified as described herein (or population of such Tregs).

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

In some embodiments of the invention, FOXP3 expression is increased by introducing into the isolated Tregs a polynucleotide encoding a FOXP3 polypeptide.

The term “introduce” refers to methods for inserting foreign DNA into a cell, including both transfection and transduction methods. Transfection is the process of introducing nucleic acids into a cell by non-viral methods. Transduction is the process of introducing foreign DNA into a cell via a viral vector. Particularly, the introduction is carried out prior to exposure of the Treg to proinflammatory conditions.

A “FOXP3 polypeptide” is a polypeptide having FOXP3 activity i.e. a polypeptide able to bind FOXP3 target DNA and function as a transcription factor regulating development and function of Tregs. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference).

### Polynucleotides and Polypeptides

The terms “polynucleotide” and “nucleic acid” are intended to be synonymous with each other. A polynucleotide may be any suitable type of nucleotide sequence, such as a synthetic RNA/DNA sequence, a cDNA sequence or a partial genomic DNA sequence.

The term “polypeptide” is synonymous with “protein” and means a series of residues, typically L-amino acids, connected to one another typically by peptide bonds between the α-amino and carboxyl groups of adjacent amino acids.

Numerous different polynucleotides can encode the same polypeptide as a result of the degeneracy of the genetic code. The skilled person may make nucleotide substitutions that do not affect the polypeptide sequence encoded by the polynucleotides to reflect the codon usage of any particular host organism in which the polypeptides are to be expressed.

The polynucleotide may comprise DNA or RNA, may be single-stranded or double-stranded and may include synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. Polynucleotides may be modified by any method in the art. Such modifications may enhance the in vivo activity or life span of the polynucleotide.

The polynucleotide may be in isolated or recombinant form. It may be incorporated into a vector and the vector may be incorporated into a host cell.

The polynucleotide may be codon optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. Suitably, the polynucleotide may be codon optimised for expression in a murine model of disease. Suitably, the polynucleotide may be codon optimised for expression in a human subject.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms. Codon optimisation may also involve the removal of mRNA instability motifs and cryptic splice sites.

### Foxp3 Polypeptide Sequences

Suitably, the FOXP3 polypeptide may comprise the polypeptide sequence of a human FOXP3, such as UniProtKB accession Q9BZS1, or a functional fragment thereof:

(SEQ ID NO: 3)
MPNPRPGKPSAPSLALGPSPGASPSWRAAPKASDLLGARGPGGTF
QGRDLRGGAHASSSSLNPMPPSQLQLPTLPLVMVAPSGARLGPLP
HLQALLQDRPHFMHQLSTVDAHARTPVLQVHPLESPAMISLTPPT
TATGVFSLKARPGLPPGINVASLEWVSREPALLCTFPNPSAPRKD
STLSAVPQSSYPLLANGVCKWPGCEKVFEEPEDFLKHCQADHLLD
EKGRAQCLLQREMVQSLEQQLVLEKEKLSAMQAHLAGKMALTKAS
SVASSDKGSCCIVAAGSQGPVVPAWSGPREAPDSLFAVRRHLWGS
HGNSTFPEFLHNMDYFKFHNMRPPFTYATLIRWAILEAPEKQRTL
NEIYHWFTRMFAFFRNHPATWKNAIRHNLSLHKCFVRVESEKGAV
WTVDELEFRKKRSQRPSRCSNPTPGP

In some embodiments of the invention, the FOXP3 polypeptide comprises an amino acid sequence which is at least 80% identical to SEQ ID NO: 3 or a functional fragment thereof. Suitably, the FOXP3 polypeptide comprises an amino acid sequence which is at least 85, 90, 95, 98 or 99% identical to SEQ ID NO: 3 or a functional fragment thereof. In some embodiments, the FOXP3 polypeptide comprises SEQ ID NO: 3 or a functional fragment thereof.

Suitably, the FOXP3 polypeptide may be a variant of SEQ ID NO: 3, for example a natural variant. Suitably, the FOXP3 polypeptide is an isoform of SEQ ID NO: 3. For example, the FOXP3 polypeptide may comprise a deletion of amino acid positions 72-106 relative to SEQ ID NO: 3. Alternatively, the FOXP3 polypeptide may comprise a deletion of amino acid positions 246-272 relative to SEQ ID NO: 3.

Suitably, the FOXP3 polypeptide comprises SEQ ID NO: 4 or a functional fragment thereof:

(SEQ ID NO: 4)
MPNPRPGKPSAPSLALGPSPGASPSWRAAPKASDLLGARGPGGTF
QGRDLRGGAHASSSSLNPMPPSQLQLPTLPLVMVAPSGARLGPLP
HLQALLQDRPHFMHQLSTVDAHARTPVLQVHPLESPAMISLTPPT
TATGVFSLKARPGLPPGINVASLEWVSREPALLCTFPNPSAPRKD
STLSAVPQSSYPLLANGVCKWPGCEKVFEEPEDFLKHCQADHLLD
EKGRAQCLLQREMVQSLEQVEELSAMQAHLAGKMALTKASSVASS
DKGSCCIVAAGSQGPVVPAWSGPREAPDSLFAVRRHLWGSHGNST
FPEFLHNMDYFKFHNMRPPFTYATLIRWAILEAPEKQRTLNEIYH
WFTRMFAFFRNHPATWKNAIRHNLSLHKCFVRVESEKGAVWTVDE
LEFRKKRSQRPSRCSNPTPGPEGRGSLLTCGDVEEN.

Suitably the FOXP3 polypeptide comprises an amino acid sequence which is at least 80% identical to SEQ ID NO: 4 or a functional fragment thereof. Suitably, the polypeptide comprises an amino acid sequence which is 85, 90, 95, 98 or 99% identical to SEQ ID NO: 4 or a functional fragment thereof.

Suitably, the FOXP3 polypeptide may be a variant of SEQ ID NO: 4, for example a natural variant. Suitably, the FOXP3 polypeptide is an isoform of SEQ ID NO: 4 or a functional fragment thereof. For example, the FOXP3 polypeptide may comprise a deletion of amino acid positions 72-106 relative to SEQ ID NO: 4. Alternatively, the FOXP3 polypeptide may comprise a deletion of amino acid positions 246-272 relative to SEQ ID NO: 4.

### FOXP3 Polynucleotide Sequences

Suitably, the FOXP3 polypeptide is encoded by the polynucleotide sequence set forth in SEQ ID NO: 1:

(SEQ ID NO: 1)
ATGCCCAACCCCAGGCCTGGCAAGCCCTCGGCCCCTTCCTTGGCC
CTTGGCCCATCCCCAGGAGCCTCGCCCAGCTGGAGGGCTGCACCC
AAAGCCTCAGACCTGCTGGGGGCCCGGGGCCCAGGGGGAACCTTC
CAGGGCCGAGATCTTCGAGGCGGGGCCCATGCCTCCTCTTCTTCC
TTGAACCCCATGCCACCATCGCAGCTGCAGCTGCCCACACTGCCC
CTAGTCATGGTGGCACCCTCCGGGGCACGGCTGGGCCCCTTGCCC
CACTTACAGGCACTCCTCCAGGACAGGCCACATTTCATGCACCAG
CTCTCAACGGTGGATGCCCACGCCCGGACCCCTGTGCTGCAGGTG
CACCCCCTGGAGAGCCCAGCCATGATCAGCCTCACACCACCCACC
ACCGCCACTGGGGTCTTCTCCCTCAAGGCCCGGCCTGGCCTCCCA
CCTGGGATCAACGTGGCCAGCCTGGAATGGGTGTCCAGGGAGCCG
GCACTGCTCTGCACCTTCCCAAATCCCAGTGCACCCAGGAAGGAC
AGCACCCTTTCGGCTGTGCCCCAGAGCTCCTACCCACTGCTGGCA
AATGGTGTCTGCAAGTGGCCCGGATGTGAGAAGGTCTTCGAAGAG
CCAGAGGACTTCCTCAAGCACTGCCAGGCGGACCATCTTCTGGAT
GAGAAGGGCAGGGCACAATGTCTCCTCCAGAGAGAGATGGTACAG
TCTCTGGAGCAGCAGCTGGTGCTGGAGAAGGAGAAGCTGAGTGCC
ATGCAGGCCCACCTGGCTGGGAAAATGGCACTGACCAAGGCTTCA
TCTGTGGCATCATCCGACAAGGGCTCCTGCTGCATCGTAGCTGCT
GGCAGCCAAGGCCCTGTCGTCCCAGCCTGGTCTGGCCCCCGGGAG
GCCCCTGACAGCCTGTTTGCTGTCCGGAGGCACCTGTGGGGTAGC
CATGGAAACAGCACATTCCCAGAGTTCCTCCACAACATGGACTAC
TTCAAGTTCCACAACATGCGACCCCCTTTCACCTACGCCACGCTC
ATCCGCTGGGCCATCCTGGAGGCTCCAGAGAAGCAGCGGACACTC
AATGAGATCTACCACTGGTTCACACGCATGTTTGCCTTCTTCAGA
AACCATCCTGCCACCTGGAAGAACGCCATCCGCCACAACCTGAGT
CTGCACAAGTGCTTTGTGCGGGTGGAGAGCGAGAAGGGGGCTGTG
TGGACCGTGGATGAGCTGGAGTTCCGCAAGAAACGGAGCCAGAGG
CCCAGCAGGTGTTCCAACCCTACACCTGGCCCCTGA

In some embodiments of the invention, the polynucleotide encoding the FOXP3 polypeptide or variant comprises a polynucleotide sequence which is at least 80% identical to SEQ ID NO: 1 or a functional fragment thereof. Suitably, the polynucleotide encoding the FOXP3 polypeptide or variant comprises a polynucleotide sequence which is at least 85, 90, 95, 98 or 99% identical to SEQ ID NO: 1 or a functional fragment thereof. In some embodiments of the invention, the polynucleotide encoding the FOXP3 polypeptide or variant comprises SEQ ID NO: 1 or a functional fragment thereof.

Suitably, the FOXP3 polypeptide is encoded by the polynucleotide sequence set forth in SEQ ID NO: 2:

(SEQ ID NO: 2)
GAATTCGTCGACATGCCCAACCCCAGACCCGGCAAGCCTTCTGCC
CCTTCTCTGGCCCTGGGACCATCTCCTGGCGCCTCCCCATCTTGG
AGAGCCGCCCCTAAAGCCAGCGATCTGCTGGGAGCTAGAGGCCCT
GGCGGCACATTCCAGGGCAGAGATCTGAGAGGCGGAGCCCACGCC
TCTAGCAGCAGCCTGAATCCCATGCCCCCTAGCCAGCTGCAGCTG
CCTACACTGCCTCTCGTGATGGTGGCCCCTAGCGGAGCTAGACTG
GGCCCTCTGCCTCATCTGCAGGCTCTGCTGCAGGACCGGCCCCAC
TTTATGCACCAGCTGAGCACCGTGGACGCCCACGCCAGAACACCT
GTGCTGCAGGTGCACCCCCTGGAAAGCCCTGCCATGATCAGCCTG
ACCCCTCCAACCACAGCCACCGGCGTGTTCAGCCTGAAGGCCAGA
CCTGGACTGCCCCCTGGCATCAATGTGGCCAGCCTGGAATGGGTG
TCCCGCGAACCTGCCCTGCTGTGCACCTTCCCCAATCCTAGCGCC
CCCAGAAAGGACAGCACACTGTCTGCCGTGCCCCAGAGCAGCTAT
CCCCTGCTGGCTAACGGCGTGTGCAAGTGGCCTGGCTGCGAGAAG
GTGTTCGAGGAACCCGAGGACTTCCTGAAGCACTGCCAGGCCGAC
CATCTGCTGGACGAGAAAGGCAGAGCCCAGTGCCTGCTGCAGCGC
GAGATGGTGCAGTCCCTGGAACAGCAGCTGGTGCTGGAAAAAGAA
AAGCTGAGCGCCATGCAGGCCCACCTGGCCGGAAAGATGGCCCTG
ACAAAAGCCAGCAGCGTGGCCAGCTCCGACAAGGGCAGCTGTTGT
ATCGTGGCCGCTGGCAGCCAGGGACCTGTGGTGCCTGCTTGGAGC
GGACCTAGAGAGGCCCCCGATAGCCTGTTTGCCGTGCGGAGACAC
CTGTGGGGCAGCCACGGCAACTCTACCTTCCCCGAGTTCCTGCAC
AACATGGACTACTTCAAGTTCCACAACATGAGGCCCCCCTTCACC
TACGCCACCCTGATCAGATGGGCCATTCTGGAAGCCCCCGAGAAG
CAGCGGACCCTGAACGAGATCTACCACTGGTTTACCCGGATGTTC
GCCTTCTTCCGGAACCACCCCGCCACCTGGAAGAACGCCATCCGG
CACAATCTGAGCCTGCACAAGTGCTTCGTGCGGGTGGAAAGCGAG
AAGGGCGCCGTGTGGACAGTGGACGAGCTGGAATTTCGGAAGAAG
CGGTCCCAGAGGCCCAGCCGGTGTAGCAATCCTACACCTGGCCCT
GAGGGCAGAGGAAGTCTGCTAACATGCGGTGACGTCGAGGAGAAT
CC.

In some embodiments of the invention, the polynucleotide encoding the FOXP3 polypeptide or variant comprises a polynucleotide sequence which is at least 80% identical to SEQ ID NO: 2 or a functional fragment thereof. Suitably, the polynucleotide encoding the FOXP3 polypeptide or variant comprises a polynucleotide sequence which is at least 85, 90, 95, 98 or 99% identical to SEQ ID NO: 2 or a functional fragment thereof. In some embodiments of the invention, the polynucleotide encoding the FOXP3 polypeptide or variant comprises SEQ ID NO: 2 or a functional fragment thereof.

Suitably, the polynucleotide encoding the FOXP3 polypeptide or variant thereof may be codon optimised. Suitably, the polynucleotide encoding the FOXP3 polypeptide or variant thereof may be codon optimised for expression in a human cell.

### Sequence Comparisons

Sequence comparisons can be conducted by eye, or more usually, with the aid of readily available sequence comparison programs. These publicly and commercially available computer programs can calculate sequence identity between two or more sequences.

Sequence identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible (reflecting higher relatedness between the two compared sequences) will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % sequence identity therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387 incorporated herein by reference). Examples of other software than can perform sequence comparisons include, but are not limited to, the BLAST package (see Ausubel et al., 1999 ibid—Chapter 18), FASTA (Atschul et al., 1990, J. Mol. Biol., 403-410 incorporated herein by reference) and the GENEWORKS suite of comparison tools. Both BLAST and FASTA are available for offline and online searching (see Ausubel et al., 1999 ibid, pages 7-58 to 7-60 incorporated herein by reference). However it is preferred to use the GCG Bestfit program.

Suitably, the sequence identity may be determined across the entirety of the sequence. Suitably, the sequence identity may be determined across the entirety of the candidate sequence being compared to a sequence recited herein.

Although the final sequence identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see user manual for further details). Preferably, the public default values for the GCG package, or in the case of other software the default matrix, such as BLOSUM62, are used.

Once the software has produced an optimal alignment, it is possible to calculate % sequence identity. The software typically does this as part of the sequence comparison and generates a numerical result.

### Vector

In some embodiments of the invention the polynucleotide encoding FOXP3 is a contiguous portion of an expression vector.

The term “expression vector” means a construct enabling expression of the FOXP3 polypeptide. Suitably, the expression vector is a cloning vector.

Suitable vectors may include, but are not limited to, plasmids, viral vectors, transposons, or nucleic acid complexed with polypeptide or immobilised onto a solid phase particle.

Preferably, the expression vector is capable of sustained high-level expression in host cells.

The expression vector may be a retroviral vector. The expression vector may be based on or derivable from the MP71 vector backbone. The expression vector may lack a full-length or truncated version of the Woodchuck Hepatitis Response Element (WPRE).

In some embodiments of the invention the vector also encodes a T cell receptor (TCR).

A TCR is a cell surface molecule that binds fragments of antigen bound to major histocompatibility complex (MHC) molecules on antigen presenting cells as part of directing an immune response. Suitably, the TCR may be a recombinant protein, in other words the TCR may be an exogenous protein which is not naturally expressed by the present Treg of the invention.

In some embodiments of the invention the vector also encodes a chimeric antigen receptor (CAR).

A CAR is a recombinant cell surface molecule expressed by an engineered T cell that binds antigen expressed on the surface of other cells as part of directing an immune response. More particularly, CARs are proteins which graft the specificity of an antigen binder, such as a monoclonal antibody (mAb), to the effector function of a T-cell. Their usual form is that of a type I transmembrane domain protein with an antigen recognizing amino terminus, a spacer, a transmembrane domain all connected to a compound endodomain which transmits T-cell survival and activation signals.

Where the vector comprises a polynucleotide encoding a TCR or CAR (e.g. an anti HLA-A2 CAR) in addition to a polynucleotide encoding FOXP3; the vector may have the orientation of: 5′ FOXP3−TCR/CAR 3′. Accordingly the polynucleotide encoding a FOXP3 may be 5′ to the polynucleotide encoding CAR or TCR.

Suitably, the polynucleotide encoding FOXP3 may be separated from the polynucleotide encoding a TCR or CAR by a nucleic acid sequence which enables both the nucleic acid sequence encoding FOXP3 and the nucleic acid sequence encoding the TCR or CAR to be expressed from the same mRNA transcript.

For example, the polynucleotide may comprise an internal ribosome entry site (IRES) between the nucleic acid sequences which encode (i) FOXP3 and (ii) the TCR or CAR. An IRES is a nucleotide sequence that allows for translation initiation in the middle of a mRNA sequence.

The polynucleotide may comprise a nucleic acid sequence encoding (i) FOXP3 and (ii) the TCR or CAR linked by an internal self-cleaving sequence.

Suitably, the vector may have the structure: 5′ Strong promoter (e.g. LTR)-FoxP3-2A-CAR/TCR-3′LTR. Here, FOXP3 expression is directly driven by the strong LTR promoter for optimal expression. CAR/TCR is preceded by a 2A sequence and expression of the CAR/TCR is thus dependent on both LTR promoter activity and 2A cleavage activity. Importantly, a configuration in which FOXP3 precedes CAR/TCR in the 5′ to 3′ direction ensures that CAR/TCR expression can only occur when FOXP3 has been expressed and that expression of CAR/TCR without FOXP3 does not occur. This is a particular advantage in the present context of an engineered Treg, as it reduces the risk of an engineered Treg acquiring an effector phenotype and/or reduces the risk associated with introducing the CAR or TCR into a T effector cell present in a starting population.

The internal self-cleaving sequence may be any sequence which enables the polypeptide comprising (i) FOXP3 and (ii) the TCR or CAR to become separated.

The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide, various models have been proposed for to account for the “cleavage” activity: proteolysis by a host-cell proteinase, autoproteolysis or a translational effect (Donnelly et al (2001) J. Gen. Virol. 82:1027-1041 incorporated herein by reference). The exact mechanism of such “cleavage” is not important for the purposes of the present invention, as long as the cleavage site, when positioned between nucleic acid sequences which encode proteins, causes the proteins to be expressed as separate entities.

The self-cleaving peptide may be a 2A self-cleaving peptide from an aphtho- or a cardiovirus.

A variant can be considered in terms of similarity (i.e. amino acid residues having similar chemical properties/functions), preferably a variant is expressed in terms of sequence identity.

Sequence comparisons can be conducted by eye, or more usually, with the aid of readily available sequence comparison programs. These publicly and commercially available computer programs can calculate sequence identity between two or more sequences.

Suitably, the FOXP3 polypeptide expressed from the present vector may be positioned at the N-terminal of a self-cleaving peptide, for example a 2A self-cleaving peptide. Such a FOXP3-2A polypeptide may comprise a sequence shown as SEQ ID NO: 5 or 6; or a variant of SEQ ID NO: 5 or 6 which is at least 80% identical thereto. Suitably, the variant may be at least 85, 90, 95, 98 or 99% identical to SEQ ID NO: 5 or 6.

SEQ ID NO: 5
MPNPRPGKPSAPSLALGPSPGASPSWRAAPKASDLLGARGPGGTF
QGRDLRGGAHASSSSLNPMPPSQLQLPTLPLVMVAPSGARLGPLP
HLQALLQDRPHFMHQLSTVDAHARTPVLQVHPLESPAMISLTPPT
TATGVFSLKARPGLPPGINVASLEWVSREPALLCTFPNPSAPRKD
STLSAVPQSSYPLLANGVCKWPGCEKVFEEPEDFLKHCQADHLLD
EKGRAQCLLQREMVQSLEQQLVLEKEKLSAMQAHLAGKMALTKAS
SVASSDKGSCCIVAAGSQGPVVPAWSGPREAPDSLFAVRRHLWGS
HGNSTFPEFLHNMDYFKFHNMRPPFTYATLIRWAILEAPEKQRTL
NEIYHWFTRMFAFFRNHPATWKNAIRHNLSLHKCFVRVESEKGAV
WTVDELEFRKKRSQRPSRCSNPTPGPGATNFSLLKQAGDVEENPG
PS
SEQ ID NO: 6
MPNPRPGKPSAPSLALGPSPGASPSWRAAPKASDLLGARGPGGTF
QGRDLRGGAHASSSSLNPMPPSQLQLPTLPLVMVAPSGARLGPLP
HLQALLQDRPHFMHQLSTVDAHARTPVLQVHPLESPAMISLTPPT
TATGVFSLKARPGLPPGINVASLEWVSREPALLCTFPNPSAPRKD
STLSAVPQSSYPLLANGVCKWPGCEKVFEEPEDFLKHCQADHLLD
EKGRAQCLLQREMVQSLEQVEELSAMQAHLAGKMALTKASSVASS
DKGSCCIVAAGSQGPVVPAWSGPREAPDSLFAVRRHLWGSHGNST
FPEFLHNMDYFKFHNMRPPFTYATLIRWAILEAPEKQRTLNEIYH
WFTRMFAFFRNHPATWKNAIRHNLSLHKCFVRVESEKGAVWTVDE
LEFRKKRSQRPSRCSNPTPGPEGRGSLLTCGDVEENGATNFSLLK
QAGDVEENPGPS

### Viral Transduction

In some embodiments of the invention, the polynucleotide encoding FOXP3 is introduced into the isolated Tregs by viral transduction.

Viral delivery systems include but are not limited to adenovirus vector, an adeno-associated viral (AAV) vector, a herpes viral vector, retroviral vector, lentiviral vector, baculoviral vector.

In some embodiments, the polynucleotide encoding FOXP3 is introduced into the isolated Tregs by retroviral transduction.

Retroviruses are RNA viruses with a life cycle different to that of lytic viruses. In this regard, a retrovirus is an infectious entity that replicates through a DNA intermediate. When a retrovirus infects a cell, its genome is converted to a DNA form by a reverse transcriptase enzyme. The DNA copy serves as a template for the production of new RNA genomes and virally encoded proteins necessary for the assembly of infectious viral particles.

There are many retroviruses, for example murine leukemia virus (MLV), human immunodeficiency virus (HIV), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses.

A detailed list of retroviruses may be found in Coffin et al. (“Retroviruses” 1997 Cold Spring Harbour Laboratory Press Eds: J M Coffin, SM Hughes, HE Varmus pp 758-763, incorporated herein by reference).

Lentiviruses also belong to the retrovirus family, but they can infect both dividing and non-dividing cells (Lewis et al. 1992 EMBO J. 3053-3058, incorporated herein by reference).

For efficient infection of human cells, viral particles may be packaged with amphotropic envelopes or gibbon ape leukemia virus envelopes.

### Isolating Tregs

In some embodiments, the method according to the invention comprises:
(a) isolating the Treg from a cell population; and
(b) increasing FOXP3 expression in the Treg.

Suitably, exogenous FOXP3 expression in the Treg is increased, by introducing an exogenous polynucleotide encoding a FOXP3 polypeptide.

The expression “isolating the Treg from a cell population” means to separate out the Treg from a heterogeneous mixture of multiple different types of cells. Suitably the cell population is from a sample from a human subject.

Suitably, the Treg is isolated as a population of Tregs.

Suitably, the population of Tregs comprises at least 70% Tregs, such as 75%, 85%, 90% or 95% Tregs.

In some embodiments of the invention, the cell population comprises or consists of peripheral blood mononuclear cells (PBMCs).

A PBMC is any blood cell with a round nucleus found within the circulating pool of blood, rather than sequestered in the bone marrow, liver, spleen or lymphatic system. PBMCs consist of monocytes and lymphocytes (T cells, B cells and NK cells). Techniques for isolation of PBMCs from whole blood are known in the art. For example, PBMCs can be separated from a blood sample by addition of a density gradient medium, such as Ficoll (GE Healthcare), followed by centrifugation. The different types of cells in the blood separate out into different layers, including a layer containing the PBMCs.

In some embodiments of the invention, isolating the Treg comprises isolating CD4⁺ T cells. In some embodiments, isolating the Treg comprises isolating CD4⁺ T cells and subsequently isolating the Treg from the CD4⁺ T cells.

CD4 (cluster of differentiation 4) is a co-receptor of the T cell receptor expressed by various types of T cells. Isolation of CD4⁺ cells separates T cells, including Tregs, from the initial cell population. The Tregs may then be isolated from this T cell-enriched population.

Techniques for isolating specific cell types from a heterogeneous population of cells are known in the art. Examples include use of immuno-magnetic beads and fluorescence-activated cell sorting.

In some embodiments of the invention, isolating the population of Tregs comprises using immuno-magnetic beads. Various companies (e.g. Miltenyi Biotec, Stem Cell Technologies, ThermoFisher Scientific) offer kits comprising immuno-magnetic beads for isolation of specific types of T cells (see, for example, Fallarino et al. (2003) Modulation of tryptophan catabolism by regulatory T cells. Nat. Immunol. 4: 1206-1212, incorporated herein by reference). These isolation kits make use of antibodies widely available in the art to T cell surface proteins such as CD8, CD25, CD49b and others. For example, CD4⁺ cells may be first negatively selected by incubating the cell population with biotin-conjugated antibodies to markers of non-CD4⁺ cells (e.g. CD8) and removing these cells using anti-biotin magnetic beads. Then, Tregs may be positively selected by incubation with anti-CD25-labelled beads.

In some embodiments of the invention, isolating the population of Tregs comprises fluorescence-activated cell sorting (FACS). In some embodiments, the Tregs are sorted according to their CD4⁺CD25^{hi}CD127⁻ phenotype.

Natural Tregs may be sorted from induced Tregs on the basis of expression of Helios protein or Neuropilin 1. In some embodiments of the invention, the natural Tregs may be sorted according to their CD4⁺CD25⁺FOXP3⁺Helios⁺Neuropilin1⁺ phenotype.

FACS is a form of flow cytometry which is well-known in the art. During FACS, cells are suspended in fluid and streamed through a detection system that analyses various characteristics. Cells can be sorted according to their characteristics using this method. In particular, in FACS, molecules are marked using fluorescent antibodies and cells sorted according to their degree of fluorescence, which indicates level of expression of the particular molecule (see Adan et al. Flow cytometry: basic principles and applications Crit. Rev. Biotechnol. 2017 March; 37(2):163-176, incorporated herein by reference).

### Engineered Tregs

The methods of the invention provide a Treg with a higher FOXP3 expression than a corresponding, non-engineered Treg which is capable of maintaining its suppressive function when exposed to proinflammatory conditions.

“Higher FOXP3 expression” means levels of FOXP3 mRNA or protein in the engineered Treg are higher than they were before the Treg was manipulated by human intervention to alter its gene expression.

The “higher FOXP3 expression” may be defined and determined as described herein.

Suitably, the level of CD25 mRNA and/or protein in a Treg modified as described herein (or a population of such Tregs) may be increased to at least 1.5-fold greater than the level in a corresponding Treg which has not been modified as described herein (or population of such Tregs).

Suitably, the level of CD25 mRNA and/or protein in a Treg modified as described herein (or a population of such Tregs) may be increased to at least 2-fold greater than the level in a corresponding Treg which has not been modified as described herein (or population of such Tregs).

Suitably, the level of CD25 mRNA and/or protein in a Treg modified as described herein (or a population of such Tregs) may be increased to at least 5-fold greater than the level in a corresponding Treg which has not been modified as described herein (or population of such Tregs).

Suitably, the level of CTLA-4 mRNA and/or protein in a Treg modified as described herein (or a population of such Tregs) may be increased to at least 1.5-fold greater than the level in a corresponding Treg which has not been modified as described herein (or population of such Tregs).

Suitably, the level of CTLA-4 mRNA and/or protein in a Treg modified as described herein (or a population of such Tregs) may be increased to at least 2-fold greater than the level in a corresponding Treg which has not been modified as described herein (or population of such Tregs).

Suitably, the level of CTLA-4 mRNA and/or protein in a Treg modified as described herein (or a population of such Tregs) may be increased to at least 5-fold greater than the level in a corresponding Treg which has not been modified as described herein (or population of such Tregs).

In some embodiments of the invention, the engineered Treg comprises an exogenous polynucleotide encoding a FOXP3 polypeptide.

An “exogenous polynucleotide” is a polynucleotide that originates outside the Treg. The exogenous polynucleotide may be introduced into the Treg as part of an expression vector. Accordingly, the exogenous polynucleotide may be contiguous with expression vector elements, such as a promoter.

In some embodiments of the invention, the FOXP3 polypeptide comprises an amino acid sequence which is at least 80% identical to SEQ ID NO: 3 or 4 or a functional fragment thereof. Suitably, the FOXP3 polypeptide comprises an amino acid sequence which is at least 85, 90, 95, 98 or 99% identical to SEQ ID NO: 3 or 4 or a functional fragment thereof. In some embodiments, the FOXP3 polypeptide comprises SEQ ID NO: 3 or 4 or a functional fragment thereof.

In some embodiments of the invention, the exogenous polynucleotide encoding FOXP3 comprises a polynucleotide sequence which is at least 80% identical to SEQ ID NO: 1 or 2.

In some embodiments of the invention, the polynucleotide encoding FOXP3 is identical to SEQ ID NO: 1 or 2.

In some embodiments of the invention, the exogenous polynucleotide encoding FOXP3 is a contiguous portion of a vector.

In some embodiments of the invention, the vector also encodes a T cell receptor (TCR) or a CAR, particularly an anti-HLA A2 CAR.

In some embodiments of the invention, the vector comprises a polynucleotide sequence which is at least 80% identical to SEQ ID NO: 5. In some embodiments of the invention, the vector comprises a polynucleotide sequence identical to SEQ ID NO: 5.

As such, the invention provides an engineered Treg expressing exogenous FOXP3 obtainable or obtained by the method of the invention, wherein said Treg can maintain its suppressive function when exposed to proinflammatory conditions.

The engineered Treg may be obtained by a process comprising:
(i) isolating a Treg from a cell population; and
(ii) introducing a polynucleotide encoding an exogenous FOXP3 polypeptide into the isolated Treg to maintain the ability of the Treg to suppress immune responses under proinflammatory conditions.

The Treg may be a CD4+CD25+CD127−/lowCD45RA+ Treg.

In addition, the invention provides a method of making an engineered Treg as disclosed herein comprising the step of introducing into a T regulatory cell or a pluripotent or multipotent cell, one or more polynucleotide sequences encoding exogenous FOXP3, a CAR or exogenous TCR, wherein when one or more polynucleotide sequences are introduced into a pluripotent or multipotent cell, the method includes a subsequent step of differentiating the cell to a T regulatory cell.

The T regulatory cell may be isolated from a subject, particularly by apheresis and subsequent enrichment; or the T regulatory cell may be obtained by differentiation of a pluripotent cell, particularly an iPSC.

### Compositions

The invention also provides a pharmaceutical composition comprising an engineered Treg obtainable or obtained by a method of the invention.

Such pharmaceutical composition may comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as (or in addition to) the carrier, excipient or diluent, any suitable binder(s), lubricant(s), suspending agent(s), coating agent(s), solubilising agent(s) and other carrier agents.

The pharmaceutical compositions typically should be sterile and stable under the conditions of manufacture and storage. Formulations for parenteral administration include, but are not limited to, suspensions, solutions, emulsions in oily or aqueous vehicles, pastes, and implantable sustained-release or biodegradable formulations. Sterile injectable formulations may be prepared using a non-toxic parenterally acceptable diluent or solvent. A pharmaceutical composition of the present invention may include pharmaceutically acceptable dispersing agents, wetting agents, suspending agents, isotonic agents, coatings, antibacterial and antifungal agents, carriers, excipients, salts, or stabilizers which are non-toxic to the subjects at the dosages and concentrations employed. Preferably, such a composition can further comprise a pharmaceutically acceptable carrier or excipient for use in the treatment of disease that that is compatible with a given method and/or site of administration, for instance for parenteral (e.g. sub-cutaneous, intradermal, or intravenous injection) or intrathecal administration.

The composition may be produced using current good manufacturing practices (cGMP).

Suitably the pharmaceutical composition comprising an engineered Treg may comprise an organic solvent, such as but not limited to, methyl acetate, dimethyl sulfoxide (DMSO), N,N-dimethylformamide (DMF), dimethoxyethane (DME), and dimethylacetamide, including mixtures or combinations thereof.

Suitably the pharmaceutical composition is endotoxin free.

Suitably, the invention provides a pharmaceutical composition comprising an engineered T cell (Treg) with the ability to suppress immune responses under proinflammatory conditions, obtained by a process comprising:
(iii) isolating a Treg from a cell population; and
(iv) introducing a polynucleotide encoding an exogenous FOXP3 polypeptide into the isolated Treg to maintain the ability of the Treg to suppress immune responses under proinflammatory conditions.

The Treg may be a CD4+CD25+CD127−/lowCD45RA+ Treg.

Prevention and/or Treatment of a Disease

The invention also provides an engineered Treg obtainable or obtained by a method of the invention, or a pharmaceutical composition of the invention, for use in prevention and/or treatment of a disease.

The invention also provides use of an engineered Treg obtainable or obtained by a method of the invention in the manufacture of a medicament for prevention and/or treatment of a disease.

The invention also provides a method of prevention and/or treatment of a disease comprising administering to a subject an engineered Treg or a composition of the invention.

Preferably, the method of prevention and/or treatment of a disease comprises administration of a pharmaceutical composition of the present invention to a subject.

The term “treat/treatment/treating” refers to administering an engineered Treg or pharmaceutical composition of the invention to a subject having an existing disease or condition in order to lessen, reduce or improve at least one symptom associated with the disease and/or to slow down, reduce or block the progression of the disease.

“Prevention”/“preventing” (or prophylaxis) refers to delaying or preventing the onset of the symptoms of the disease. Prevention may be absolute (such that no disease occurs) or may be effective only in some individuals or for a limited amount of time.

In some embodiment of the invention, the subject of the method of the invention is a mammal, preferably a cat, dog, horse, donkey, sheep, pig, goat, cow, mouse, rat, rabbit or guinea pig. Preferably the subject is a human.

The administration of a pharmaceutical composition of the invention can be accomplished using any of a variety of routes that make the active ingredient bioavailable. For example, an engineered Treg or pharmaceutical composition can be administered intravenously, intrathecally, by oral and parenteral routes, intranasally, intraperitoneally, subcutaneously, transcutaneously or intramuscularly.

Suitably, the engineered Treg or pharmaceutical composition of the invention is administered intravenously. Suitably, the engineered Treg or pharmaceutical composition of the invention is administered intrathecally.

Typically, a physician will determine the dosage that is most suitable for an individual subject, and the dosage will vary with the age, weight and response of the particular subject. The dosage is such that it is sufficient to reduce and/or prevent disease symptoms.

The skilled person appreciates, for example, that route of delivery (e.g. oral vs. intravenous vs. subcutaneous etc.) may impact the required dosage (and vice versa). For example, where particularly high concentrations of an agent within a particular site or location are desired, focussed delivery may be preferred. Other factors to be considered when optimizing routes and/or dosing schedule for a given therapeutic regimen may include, for example, the disease being treated (e.g. type or stage etc.), the clinical condition of a subject (e.g. age, overall health etc.), the presence or absence of combination therapy, and other factors known to medical practitioners.

The dosage is such that it is sufficient to stabilise or improve symptoms of the disease.

The present invention also provides a method for treating and/or preventing a disease, which comprises the step of administering a pharmaceutical composition comprising a cell e.g. a T cell according to the invention to a subject.

Suitably, the method of prevention and/or treatment of a disease may comprise:
(i) isolation of Tregs from a subject;
(ii) introducing a polynucleotide sequence encoding a FOXP3 polypeptide into (i.e. engineering) the Tregs; and
(iii) administering the engineered Tregs to the subject.

Tregs may be isolated from a patient by taking a blood sample and isolating Tregs from it using techniques known in the art, such as those described in this specification under the heading “Isolating Tregs”.

A polynucleotide encoding a FOXP3 polypeptide may be introduced into Tregs using techniques known in the art, such as those described in this specification under the heading “Viral transduction”.

Suitably the engineered Tregs may be expanded in vitro before administration to the subject. Tregs may be expanded in vitro by culturing them in TexMACX® media.

### Disease

The disease to be treated and/or prevented by the methods and uses of the present invention may be any disease which is associated with a pathological immune response.

The disease may be, for example, a cancer, infectious disease or autoimmune disease.

In some embodiments of the invention, the disease is an autoimmune disease.

The disease may have central nervous system (CNS) involvement of systemic autoimmune and inflammatory disease such as Behçet disease, sarcoidosis, systemic lupus erythematosus, juvenile idiopathic arthritis, scleroderma, and Sjögren syndrome.

The disease may be any disease wherein MBP is an antigen e.g. where MBP is a self-antigen.

Suitably the disease may be an autoimmune and inflammatory central nervous system or peripheral nervous system disease (e.g. chronic neurodegenerative conditions).

Suitably the disease may be a chronic neurodegenerative condition such as multiple sclerosis (MS), Alzheimer's disease, Parkinson's disease, neurotropic viral infections, stroke, paraneoplastic disorders and traumatic brain injury.

The present invention further provides a method for inducing tolerance to a transplant; treating and/or preventing cellular and/or humoral transplant rejection; treating and/or preventing graft-versus-host disease (GvHD), which comprises the step of administering an engineered Treg or a pharmaceutical composition of the invention to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

In one embodiment, the subject is a transplant recipient undergoing immunosuppression therapy.

The transplant may be selected from a liver, kidney, heart, lung, pancreas, intestine, stomach, bone marrow, vascularized composite tissue graft, and skin transplant.

### Uses

The present invention further provides use of a polynucleotide encoding a FOXP3 polypeptide to maintain the ability of a regulatory T cell (Treg) to suppress immune responses under proinflammatory conditions. As discussed previously, the maintenance of the suppressive function of a Treg of the invention which has been or is exposed to proinflammatory conditions is provided by the increased expression of FOXP3 from introduction to a Treg of a polynucleotide encoding a FOXP3 polypeptide, where the maintenance of suppressive function is as compared to a corresponding Treg comprising a polynucleotide encoding a FOXP3 polypeptide but which has not been or is not being exposed to proinflammatory conditions (i.e. has only been exposed to non-inflammatory conditions). Thus, the FOXP3 encoding polynucleotide is used to maintain suppressive function of a Treg under proinflammatory conditions.

Alternatively viewed, the present invention provides use of a polynucleotide encoding a FOXP3 polypeptide to increase the ability of a regulatory T cell (Treg) to suppress immune responses under proinflammatory conditions, as compared to a Treg which does not comprise an exogenous FOXP3 polypeptide encoding polynucleotide.

Alternatively viewed, the present invention provides use of a polynucleotide encoding a FOXP3 polypeptide to prevent loss of suppressive function in a Treg cell under proinflammatory conditions.

Further, the present invention may provide a method of increasing the percentage or amount of Tregs comprising an exogenous polynucleotide encoding FOXP3 within a population of Tregs comprising exposing the population of Tregs to proinflammatory conditions.

Particularly, an increase in the percentage or amount of Tregs comprising an exogenous polynucleotide encoding FOXP3 within the population may be an increase of at least 10, 20, 30, 40, 50, 60, 70, 80 or 90% and may be measured directly by flow cytometry using an antibody which binds to FOXP3 (after permeabilization of the cells) or by any of the methods discussed previously above. Typically, Tregs which do not comprise exogenous FOXP3 have lower FOXP3 levels than transduced Tregs and lower viability under proinflammatory conditions. Exposure of a mixed population of Tregs to proinflammatory conditions can therefore be utilised to purify Tregs comprising an exogenous polynucleotide encoding FOXP3 or to increase the number or percentage of those cells present within a population. As described previously, this can be achieved by exposure to any one or more proinflammatory cytokines, but particularly may be achieved by exposure to IL1B, IL6 and/or TNFα.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation and any amino acid sequences are written left to right in amino- to carboxy-terminal orientation.

Where a range of values is provided, each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

The singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Embodiments of the invention may be combined.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist the skilled person in carrying out the invention and are not intended in any way to limit the scope of the invention.

### EXAMPLES

### Materials and Methods

### Cloning:

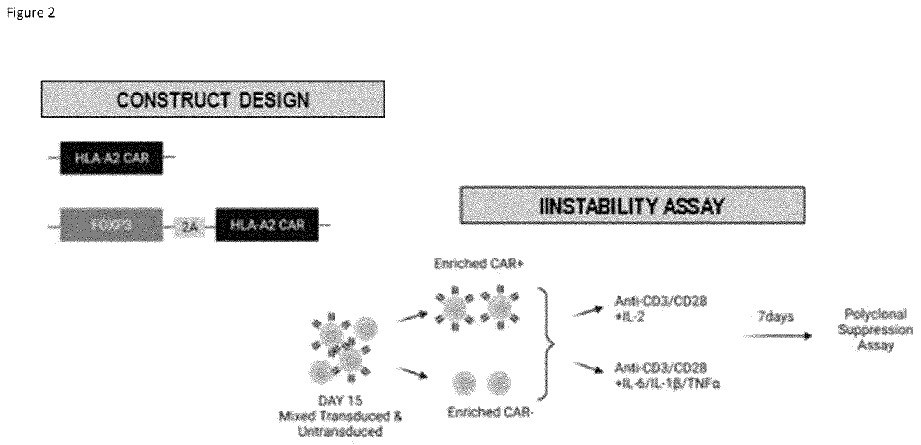

2 constructs (FIG. 2) were designed in house, and whole sequences were codon optimised for expression in human cells and manufactured. Constructs were cloned and D5a high efficiency bacteria were transformed with plasmid and grown with the selection agent ampicillin. DNA was extracted using a Miniprep Kit (Qiagen). Inserts were transferred into a lentiviral backbone by PCR cloning.

### Collection of PBMCs:

Leukocyte cones were supplied by NHS blood and transplant. PBMC were isolated using a density centrifugation protocol. Briefly, blood was diluted 1:1 with 1×PBS and layered over Ficoll-Paque (GE Healthcare). Samples were centrifuged and the leukocyte layer was removed and washed in PBS.

### Treg and Tconv Isolation Protocol:

Blood cones from HLA-A*02 negative donors were used to derive Treg populations. Blood cones were subjected to CD4 enrichment via negative selection using RosetteSep™ Human CD4+ T Cell Enrichment Cocktail. Subsequently, CD4+ cells were isolated using density centrifugation. CD4+CD25+ T cells were then isolated via positive selection using CD25 microbeads II (Miltenyi). The CD4+CD25+ fraction was stained with flow cytometry antibodies CD4 FITC (OKT4, Biolegend), CD25 PE-Cy7 (BC96, Biolegend), CD127 BV421 (A019D5, Biolegend), CD45RA BV510 (H1100, Biolegend) and the LIVE/DEAD™ Fixable Near-IR—Dead Cell Stain (Thermofisher) before FACS sorting.

### T Reg Culture Media and Expansion:

Human Regulatory T cells were cultured in Texmacs media (Miltenyi) supplemented with IL-2 and activated with Human T-Activator CD3/CD28 Dynabeads™ (Gibco). Cells were re-fed every 2 to 3 days with Treg culture media supplemented with IL-2. A second round of stimulation with Dynabeads™ was performed to promote further expansion of Treg cells.

### Transfection and Viral Particle Production

### Lentivirus:

HEK293T cells were seeded and cultured in DMEM (Dulbecco's Modified Eagle's Medium)+10% Fetal Bovine Serum (FBS) for 24 hours. Transfection reagents were brought to room temperature and were mixed with DNA construct/plasmid of interest, packaging plasmid (pD8.91) and viral envelope (pVSV-G). PEI was added to the diluted DNA and mixed and added to HEK293 Ts. Supernatant was harvested 48 hours post-transfection, filtered and virus concentrated.

### Flow Cytometry Staining to Determine CAR and RQR8 Expression

T cells were removed from culture and washed in FACS buffer and stained for HLA-A*02 specific CAR using an HLA-A*02 specific Dextramer (WB2720-APC, Immudex) in FACS buffer. Subsequently, cells were stained with the LIVE/DEAD™ Fixable Near-IR—Dead Cell Stain (Thermofisher) in PBS first and then with anti-CD4 AF700 (RPA-T4, BD), anti-CD34 FITC (QBEND/10, Thermofisher) and anti-CD3 PE-Cy7 in FACS staining buffer. For intracellular staining of FOXP3, cells were fixed and permeabilized and stained with the anti-Foxp3 PE (150D/E4, Thermofisher) antibody. Cells were analysed on a BD LSRII flow cytometer.

### Instability Assay

Frozen Tregs, transduced as described above, were thawed and incubated under two different conditions. The first condition cultured the Tregs under standard conditions as described above. The second condition involved culture of the Tregs with the additional presence of IL6 (4 ng/ml), IL1β (10 ng/ml) and TNFα (10 ng/ml). Cells were cultured for 7 days, after which a suppression assay was carried out.

### Suppression Assay

For assessing the ability of Treg to suppress effector T cell activation, Teff cells were labelled with CFSE dye. Teff cells were co-cultured with different concentrations of Treg cells (ratios Treg:Teffs of 1:1, 1:2, 1:4, 1:8, 1:16, 1:32 and 1:64. For activation, CD3/CD28 beads (1:100) were added. For CAR dependent activation, HLA A2 expressing cells are used. 72 hours after activation, cells are harvested and analysed by flow cytometry. CFSE dilution is used as a surrogate marker for Teff cell proliferation.

### Results

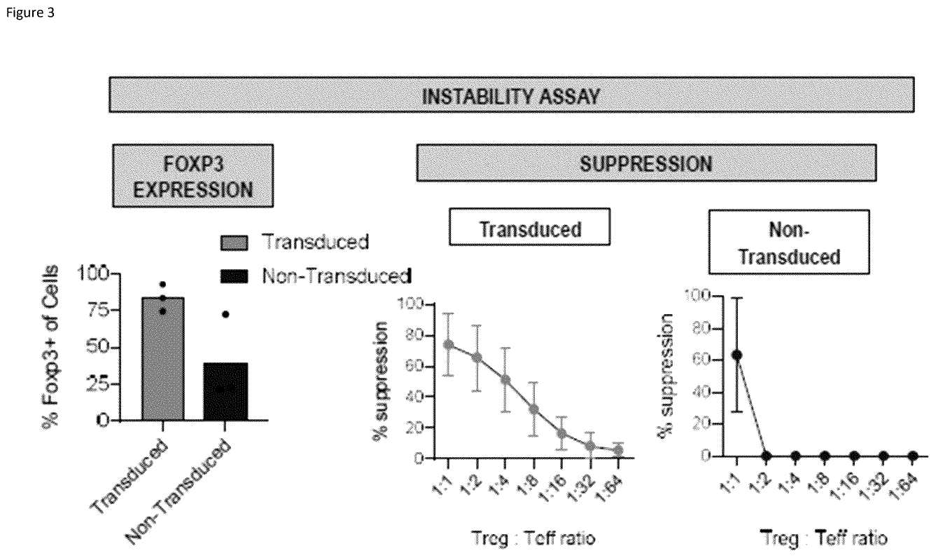

FIG. 3 shows the expression levels of FOXP3 within transduced vs non-transduced cells for Construct I (i.e. the construct expressing additional exogenous FOXP3). It can be seen that levels of FOXP3 are higher in the transduced Tregs as compared to the cells which were not transduced in the population. Thus, transducing with Construct I increases the level of FOXP3 within the cells.

Further, FIG. 3 shows the suppressive function of transduced as compared to non-transduced cells under proinflammatory conditions. Cells which express exogenous additional FOXP3 are able to maintain their suppressive function even after exposure to proinflammatory cytokines.

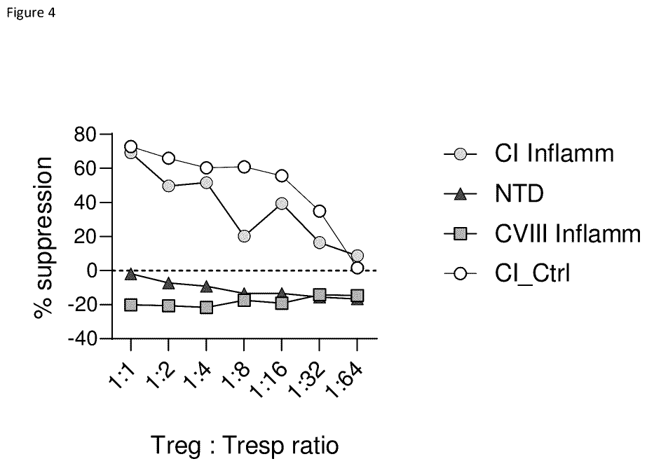

This maintenance of suppressive effect is further evidenced in FIG. 4 which shows a comparison between the suppressive function or ability of cells transduced with Construct I (having exogenous FOXP3 expression and CAR expression) and Construct VIII (expressing the same CAR as Construct I but without exogenous FOXP3 expression). Cells transduced with Construct I are able to maintain their suppressive function, as compared to cells transduced with Construct VIII which are not able to suppress after exposure to proinflammatory conditions. Cells transduced with Construct I exposed to proinflammatory conditions only show a minor decrease in suppressive function as compared to the same cells cultured under standard conditions (no proinflammatory cytokines), demonstrating how effective the presence of exogenous FOXP3 is at maintaining suppressive function under proinflammatory conditions. Given that a Treg product will need to function under proinflammatory conditions in vivo, the presence of exogenous FOXP3 is important to provide a product which can retain its functional ability.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cellular immunology or related fields are intended to be within the scope of the following claims.

1. A method for maintaining the ability of a regulatory T cell (Treg) to suppress immune responses under proinflammatory conditions comprising introducing a polynucleotide encoding a FOXP3 polypeptide into the Treg.

2. The method of claim 1, further comprising a step of incubating the Treg under proinflammatory conditions, particularly incubating with any one or more of IL-6, IFNγ and/or IL1β.

3. A method according to claim 1 or 2 wherein:
(i) the FOXP3 polypeptide comprises an amino acid sequence which is at least 80% identical to SEQ ID NO: 3 or 4 or a functional fragment thereof; or
(ii) the polynucleotide encoding the FOXP3 polypeptide comprises a polynucleotide sequence which is at least 80% identical to SEQ ID NO: 1 or 2 or a functional fragment thereof.

4. A method according to any preceding claim wherein the polynucleotide encoding FOXP3 is a contiguous portion of an expression vector.

5. A method according to any preceding claim which further comprises introducing a polynucleotide encoding an exogenous T cell receptor (TCR) or a polynucleotide encoding a chimeric antigen receptor (CAR) into the Treg.

6. A method according to claim 5 wherein the polynucleotide encoding a FOXP3 polypeptide and the polynucleotide encoding the exogenous TCR or the CAR are provided by a single expression vector.

7. A method according to claim 6 wherein the vector comprises a nucleic acid with the orientation: 5′ FOXP3−TCR/CAR 3′.

8. A method according to any preceding claim wherein the polynucleotide encoding FOXP3 is introduced into the isolated Treg by viral transduction; optionally wherein the polynucleotide encoding FOXP3 is introduced into the isolated Treg by retroviral transduction.

9. A method according to any preceding claim comprising:
(a) isolating a Treg from a cell population; and
(b) increasing FOXP3 expression in the Treg.

10. A method according to claim 9 wherein the cell population comprises or consists of peripheral blood mononuclear cells (PBMCs).

11. A method according to claim 9 or 10 wherein isolating the Treg comprises
isolating CD4⁺ T cells; and
isolating the Treg from the CD4⁺ T cells.

12. A method according to any of claims 9-11 wherein the isolation of the Treg comprises selection using immuno-magnetic beads or fluorescence-activated cell sorting (FACS).

13. A method according to any of claims 9-12 wherein the Treg is isolated by selecting for (i) CD4⁺CD25⁺CD127⁻ and/or CD4⁺CD25⁺CD127^{low} cells; (ii) CD4⁺CD25^{hi}CD127⁻ and/or CD4⁺CD25⁺CD127^{low} cells.

14. A method according to any of claims 9-13 wherein the Treg is isolated by selecting for FOXP3⁺ cells; preferably wherein the Treg is isolated by selecting for CD4⁺CD25⁺FOXP3⁺Helios⁺Neuropilin1⁺ cells.

15. An engineered Treg obtainable or obtained by the method of any of claims 1-14.

16. A pharmaceutical composition comprising an engineered Treg according to claim 15.

17. An engineered Treg according to claim 15, or a pharmaceutical composition according to claim 16, for use in prevention and/or treatment of a disease.

18. Use of an engineered Treg according to claim 15 in the manufacture of a medicament for prevention and/or treatment of a disease.

19. A method of prevention and/or treatment of a disease comprising administering to a subject an engineered Treg or a pharmaceutical composition according to any of claims 15-18.

20. An engineered Treg for use, or a pharmaceutical composition for use, according to claim 17, use of an engineered Treg according to claim 18, or a method according to claim 19, wherein the disease is an autoimmune disease; preferably wherein the disease is multiple sclerosis.

21. An engineered Treg according to claim 15 for use in prevention and/or treatment of a disease wherein the disease is transplant rejection or graft-vs-host disease.

22. Use of a polynucleotide encoding a FOXP3 polypeptide to maintain the ability of a regulatory T cell (Treg) to suppress immune responses under proinflammatory conditions.
